(19) United States
(12) Patent Application Publication
(10) Pub. No.: US 2010/0260845 A1
(43) Pub. Date: Oct. 14, 2010

(54) Biocompatible and Biodegradable Biopolymer Matrix

(75) Inventors: A. Jayakrishnan, Madras Chennai (IN); Denis Labarre, Chatenay-Malabry Cedex (FR); Alexandre Laurent, Jouy En Josas (FR); Biji Balakrishnan, Mumbai (IN); P.R. Umashankar, Trivandrum (IN)

Correspondence Address: MCANDREWS HELD & MALLOY, LTD, 500 WEST MADISON STREET, SUITE 3400, CHICAGO, IL, 60661, US

(73) Assignee: Indo-French Center for the Promotion of Advanced Research, New Delhi (IN)

(21) Appl. No.: 12/746,625

(22) PCT Filed: Dec. 8, 2008
(86) PCT No.: PCT/IN08/00818

§ 371 Date: Jun. 7, 2010

(30) Foreign Application Priority Data
Dec. 7, 2007 (IN) 2572/DEL/2007

(51) Int. Cl. A61L 24/08

(52) U.S. Cl. 424/484; 514/55

(57) ABSTRACT

The present invention provides a biodegradable biopolymer matrix for surgical and/or therapeutic use comprising chitosan hydrochloride and dextran dialdehyde is in the ratio of 1:1 to 1:2. The invention further provides a process for preparing the biopolymer matrix and a kit for a surgical and/or therapeutic use comprising the biopolymer matrix.

### FIELD OF INVENTION

The present invention relates to the preparation of a biocompatible, biodegradable biopolymer matrix based on natural polysaccharide chitosan and dextran that can be formed in situ very rapidly.

### BACKGROUND OF THE INVENTION

Over the last 40 years, the use of surgical tissue adhesives in medicine has developed considerably and its list of application is increasing tremendously. Traditionally, the repair following surgery or trauma has been dominated by use of suture, staples and wiring. The huge commercial potential for tissue adhesives has sparked a mini revolution in medical practices recently.

The term tissue adhesive is a misnomer because these materials can also function as sealants, drug delivery systems and as wound dressings. When used as a tissue sealant or fluid barrier, the main aim is to prevent fluid or gas loss from the body. As drug delivery system, tissue sealants should protect and serve as a reservoir for the bioactive agent as well as release it to tissue at the appropriate rate. In addition, it should lead to minimal responses (inflammation, toxicity, carcinogenicity, viral transmission, etc). Optimally, however, these tissue sealants should enhance the local healing process by either stimulating tissue generation or speeding up the regenerative process. Tissue adhesives based on commercial gelatin or collagen and starch has also been recently proposed in the document WO97/29715 (Fusion Medical Technologies Inc. G. Izoret, Bioadhesive; Method for preparing same and device for applying a bioadhesive, and hardening agents for a bioadhesive). These adhesives form very viscous gels which have to be heated to a high temperature, of the order of 50-80° C., in order to be applied with a syringe. Besides the risk of potential toxicity depending on the aldehyde used, these adhesives can damage the treated tissues, in particular because of their application temperature. A photocrosslinkable bioadhesive was reported by Ono et al by introducing the photolabile azide group into chitosan which was reported to provide high adhesive and air sealing strength (K Ono. Y Saito, H Yura., K Ishikwara., A Kuita., T Akaike., M Ishihara., Photocrosslinkable chitosan as a bioadhesive, J. Biomed. Mater. Res., 49: 289-295, 2000.). This also is reported to promote wound healing. But irradiation by ultraviolet light is required for curing the adhesive and this can raise serious health problems which would limit its use. Also, the presence of any uncrosslinked azide functionality can create toxic responses in the body; K Ono. et al, 2000. Tardy et al [M. Tardy, H Volckmann, J Tiollier, P Gravagna, J L Tayot, Adhesive composition with macromolecular polyaldehyde base and method for crosslinking collagen, U.S. Pat. No. 6,165,488, 2000] have described a collagen-based biological adhesive which can be prepared using a kit consisting of, for example, two separate syringes, one containing a solution of collagen (or gelatin) oxidized with sodium periodate and stored at acidic pH in frozen form at a temperature below 0° C., preferably below −20° C. and other with an aqueous alkaline solution. The mixing of the two components is ensured by a mixer connected to the two syringes, after the oxidized collagen (or gelatin) gel has been reheated to about 40° C. in order to obtain a biocompatible adhesive, whose crosslinking is accomplished in 2 to 3 minutes. Though the properties of this adhesive are advantageous in some applications, the need for a complex cold system for the distribution of this product increases its cost and makes it uncomfortable to use.

The latest of all surgical adhesives in the market is BioGlue® manufactured and marketed by Cryo Life Inc., USA. It is a two-component surgical adhesive composed of purified bovine serum albumin (BSA) and glutaraldehyde. On application, the glutaraldehyde molecules covalently bond (cross-link) the BSA molecules to each other and to the tissue proteins at the repair site, creating a flexible mechanical seal independently of the body's clotting cascade. The delivery device-mediated application is designed to provide reproducible mixing of the components in vitro.

BioGlue® begins to polymerize within 20 to 30 seconds and reaches its bonding strength within 2 minutes.

However, the adhesive has been contraindicated in several procedures. For instance, BioGlue® reinforcement has been reported to impair vascular growth and cause stricture when applied circumferentially around an aorto-aortic anastomosis. This adhesive is therefore not recommended on cardiovascular anastomoses in pediatric patients; LeMaire S A et al (LeMaire S A, Schmittling Z C, Coselli J S et al., BioGlue surgical adhesive impairs aortic growth and causes anastomotic strictures. Ann Thorac Surg. 2002, 73:1500-5). Saline supernatants from polymerized BioGlue® contained 100 to 200 μg/mL glutaraldehyde and were cytotoxic. Application of BioGlue® to lung and liver tissue evoked serious adverse effects such as high-grade inflammation, edema, and toxic necrosis; W. Furst et al, 2005 (Fürst, W., Banerjee, A., Release of Glutaraldehyde From an Albumin-Glutaraldehyde Tissue Adhesive Causes Significant In Vitro and In Vivo Toxicity, Ann Thorac Surg 2005; 79:1522-1528).

Chitin, a naturally abundant mucopolysaccharide and the supporting material of crustaceans, insects, etc., is well known to consist of 2-acetamido-2-deoxy β-1,4-glucan. Chitin is highly insoluble and can be degraded by chitinase. Chitosan is the N-deacetylated derivative of chitin. Chitosan is biodegradable and is non-toxic. Fibers made of chitin and chitosan are also used as absorbable sutures. Chitin sutures resist attack in bile, urine and pancreatic juice, which are problem areas with other absorbable sutures. Applications of chitin have been limited because of its low solubility in most common organic solvents. It is highly insoluble material resembling cellulose in its solubility and low chemical reactivity. The solvents for chitin are concentrated acid (HCl, H₂SO₄, H₃PO₄) and amide-LiCl system (N,N-dimethylacetamide-LiCl and Nmethyl-2-pyrolidone-LiCl). These solvents accompany several problems such as chain hydrolysis, removal of residual solvents and their toxicity. Commercially available chitosan is soluble in aqueous acidic media, but inherently water-insoluble at near neutral pHs. The chemical modification of chitosan provides an alternative to improve the biopolymer's water solubility; such modification might alter the biological properties of chitosan. Also, modification reactions are generally difficult owing to the lack of solubility.

Dextrans are natural molecules consisting of repeated linear units of covalently linked (1→6′) glucopyranose which are branched at the α-(1→4′) position.

Although dextran and chitosan have been used for varying applications in biomedical field, there are no reports on making an in situ polymerizing system by combining the beneficial aspects of both. This may be due to poor solubility of chitosan in aqueous medium. Also chitosan solution in dilute acids like acetic acid and hydrochloric acid offers low pH which makes the gel almost reversible and needs further treatment with reducing agents. Thus, there is a need for an improved tissue adhesive to provide an adhesive composition which does not exhibit the major disadvantages as referred above. The prior art documents do not teach the present invention.

### BRIEF DESCRIPTION OF ACCOMPANYING DRAWINGS

FIG. 1 shows change in the degree of swelling of hydrogels prepared with DDA of 5, 50 and 90% oxidation with respect to time in PBS

FIG. 2 shows internal structure of the hydrogel (lyophilized) prepared from 5% chitosan-HCl and 10% DDA of degree of oxidation 50%

FIGS. 3 (a) and (b) show the creation of rabbit liver injury and application of the test glue respectively (c) shows the application of control glue (Bioglue™).

FIG. 4 (a) shows the test glue at 14 days (b) shows the control glue (Bioglue™) at 14 days. The adhesion of liver into abdominal wall in both control and test glue was seen in all animals.

FIG. 5 shows histological section of rabbit liver injury treated with test glue at 2 weeks Histological section of rabbit liver injury treated with test glue at 2 weeks showing the area of necrosis (star). Some giant cells and macrophages are noticed.

FIG. 6 shows Creation of liver injury. The air and blood leak (arrow)

FIG. 7 (a) shows application of control glue (BioGlue) and (b) shows persistent air leak on the incision site.

FIG. 8 (a.) shows application of test glue and (b) shows complete sealing of incision site.

FIG. 9 shows aortic sealing using test glue. The complete sealing after release of clamps is noticed.

FIG. 10 shows endoluminal surface of the sealed incision at 2 weeks autopsy. The clean endoluminal surface without any thrombus is noticed. The neointimal formation across the incision site is also noticed.

FIG. 11 shows cumulative release of FITC-albumin from chitosan hydrochloride-DDA gels.

### OBJECTIVES OF THE INVENTION

Main objective of the present invention is to provide a biocompatible, biodegradable biopolymer matrix and preparation thereof, wherein the matrix can be used as surgical and/or therapeutic agent comprising a chitosan derivative and dialdehyde derivative of polysaccharide.

Another objective of the present invention is to provide a bio-adhesive which is non-toxic, biodegradable, rapidly curing with improved adhesion and mechanical strength and ease of application as a surgical glue or sealant.

Yet another aspect of the present invention is to provide a rapidly gelling two-component polymer system which when brought together into contact at the wound site, solidifies into a biodegradable gel which can function as a wound and burn dressing material.

Still another aspect of the present invention is to provide a rapidly gelling polymer system as an injectable matrix for the controlled and prolonged delivery of drugs, growth factors, therapeutic proteins and peptides.

Still yet another aspect of the present invention is to provide a rapidly gelling polymer system as an injectable plug for therapeutic embolization and chemo-embolization.

### SUMMARY OF THE INVENTION

The present invention relates to a biocompatible, biodegradable biopolymer matrix and preparation thereof, wherein the matrix can be used as surgical and/or therapeutic agent comprising a chitosan derivative and dialdehyde derivative of polysaccharide.

One aspect of the present invention is to provide a biodegradable biopolymer matrix for surgical and/or therapeutic use comprising chitosan hydrochloride and dextran dialdehyde is in the ratio of 1:1 to 1:2.

Another aspect of the present invention provides a process of preparing the biopolymer matrix, wherein the process comprising cross linking chitosan hydrochloride and DDA in the presence of phosphate buffered saline.

Another aspect of the present invention provides a kit for a surgical and/or therapeutic use comprising the biopolymer matrix.

### DESCRIPTION OF THE INVENTION

The present invention provides a biocompatible and biodegradable biopolymer matrix for surgical and/or therapeutic use; the matrix comprises a chitosan derivative and dialdehyde derivative of a polysaccharide.

Before describing the present invention in detail, it is to be understood that unless otherwise indicated this invention is not limited to particular compositional forms, crosslinking techniques, or methods of use, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise.

Unless defined otherwise, all technical and scientific terms used herein have the meaning commonly understood by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

As used herein, the terms “bioadhesive”, “biological adhesive”, “adhesive composition”, “adhesive”, “tissue adhesive”, “biopolymer matrix”, “matrix” DDA-chitosan hydrochloride adhesive”, “gel forming composition”, “crosslinked gel”, “test glue” and “gel” are used interchangeably to refer to biocompatible compositions capable of effecting temporary or permanent attachment between the surfaces of two native tissues, or between a native tissue surface and either a non-native tissue surface or a surface of a synthetic implant.

The present invention provides a biodegradable biopolymer matrix for surgical and/or therapeutic use comprising chitosan hydrochloride and dextran dialdehyde.

The present invention provides a process of preparation of biodegradable biopolymer matrix disclosed in the present invention.

The present invention discloses the process of preparation of a water soluble derivative of chitosan and employs the same to form a biodegradable, in situ forming hydrogel by crosslinking of the said chitosan with periodate-oxidized dextran which could find application as a tissue adhesive in several surgical procedures. The adhesive thus formed can also function as a hemostatic agent, as a wound dressing material, as an aneurysm filler, as an embolic agent, as a matrix for controlled and prolonged delivery of drugs and various growth factors.

One embodiment of the present invention provides biopolymer matrix, wherein said chitosan derivative and dialdehyde derivative of polysaccharide is in the ratio of 1:1 to 1:2.

One embodiment of the present invention provides biopolymer matrix, wherein chitosan hydrochloride and dextran dialdehyde is in the ratio of 1:1.

One embodiment of the present invention provides a process of preparing the biopolymer matrix, wherein the process comprising cross linking chitosan hydrochloride and DDA in the presence of phosphate buffered saline.

One embodiment of the present invention provides biopolymer matrix, wherein the chitosan derivative is a chitosan salt selected from the group consisting of chitosan acetate, chitosan lactate, chitosan sulphate and chitosan hydrochloride.

One embodiment of the present invention provides biopolymer matrix, wherein chitosan derivative is chitosan hydrochloride.

One embodiment of the present invention provides biopolymer matrix, wherein concentration of the chitosan hydrochloride is in the range of about 1% to 20%, preferably 5% to 10%.

One embodiment of the present invention provides biopolymer matrix, wherein the polysaccharide is dextran or alginic acid.

One embodiment of the present invention provides biopolymer matrix, wherein the dialdehyde derivative of polysaccharide is dextran dialdehyde (DDA).

One embodiment of the present invention provides biopolymer matrix, wherein concentration of the DDA is 1% to 10% preferably at a concentration of 10%.

One embodiment of the present invention provides biopolymer matrix, wherein the surgical and/or therapeutic use is selected from the group consisting of wound dressing, drug delivery, aneurysm filling, embolization and bioadhesion.

One embodiment of the present invention provides a biodegradable biopolymer matrix for surgical and/or therapeutic use comprising chitosan hydrochloride and dextran dialdehyde is in the ratio of 1:1 to 1:2.

One embodiment of the present invention provides a biodegradable biopolymer matrix for surgical and/or therapeutic use comprising chitosan hydrochloride and dextran dialdehyde 1:1.

The biodegradable biopolymer matrix disclosed in the present invention provides is non-cytotoxic.

One embodiment of the present invention provides use of biopolymer matrix disclosed in the present invention as a bioadhesive or a tissue sealant.

One embodiment of the present invention provides a wound dressing comprising the biopolymer matrix.

One embodiment of the present invention provides a biopolymer matrix, wherein the matrix is formed in situ in the wound bed by the crosslinking of chitosan hydrochloride and DDA in the presence of phosphate buffered saline.

One embodiment of the present invention provides a biopolymer matrix, wherein the matrix is formed in situ in the wound bed by the crosslinking of chitosan hydrochloride and DDA in the presence of phosphate buffer without saline.

One embodiment of the present invention provides a biopolymer matrix, wherein the matrix is formed in situ intramuscularly or subcutaneously by injecting chitosan hydrochloride and DDA solutions.

One embodiment of the present invention provides a biopolymer matrix, wherein the matrix is prefabricated in the form of films, sheets or foams in their dry or wet forms and applied as a wound or burn dressing.

One embodiment of the present invention provides a biopolymer matrix, wherein the matrix is loaded with antiseptics, antibiotics or antibacterial drugs.

One embodiment of the present invention provides a biopolymer matrix, wherein the matrix is loaded with any drug.

One embodiment of the present invention provides a biopolymer matrix, wherein the matrix is loaded with peptides, proteins, hormones and growth factors.

One embodiment of the present invention provides a biopolymer matrix, wherein the matrix is used for the controlled and/or sustained delivery of drugs.

One embodiment of the present invention provides a biopolymer matrix, wherein the matrix is used for aneurysm filling.

One embodiment of the present invention provides a biopolymer matrix, wherein the matrix is used as an embolic agent for blocking blood vessels.

One embodiment of the present invention provides a process for bonding biological tissues to one another or to an implant with biopolymer matrix.

One embodiment of the present invention provides the biopolymer matrix for use as surgical tissue adhesive, in particular for sealing or closing surfaces or orifices.

One embodiment of the present invention provides a biopolymer matrix for a preferably internal application in an organism, in particular in wounds.

One embodiment of the present invention provides a biopolymer matrix, wherein the matrix is used as tissue adhesive or sealant to prevent air leakage from lungs.

One embodiment of the present invention provides a biopolymer matrix, wherein the matrix is used as tissue adhesive as an adjuvant to sutures in surgical procedures.

One embodiment of the present invention provides a biopolymer matrix, wherein the matrix is used as tissue adhesive for sealing any surgical incisions to prevent blood leakage.

One embodiment of the present invention provides a biopolymer matrix, wherein the matrix is used for wound and burn dressing.

One embodiment of the present invention provides a biopolymer matrix for wound closure, preferably of internal wounds.

One embodiment of the present invention provides a biopolymer matrix for hemostasis in cases of organ resection or organ rupture.

One embodiment of the present invention provides a biopolymer matrix of a resorbable self-adhering type to human or animal tissue and essentially consisting of at least one polymer which carries free aldehyde groups and whose aldehyde groups are able to react with amino groups of the tissue, the matrix being present in a moist form, in particular a liquid or gel-like form.

One embodiment of the present invention provides a drug delivery system comprising the biopolymer matrix as disclosed in the present invention.

One embodiment of the present invention provides a bioadhesive comprising the biopolymer matrix as disclosed in the present invention.

One embodiment of the present invention provides a method of preventing tissue adhesion after surgery, the method comprising applying to a tissue surface for which non-adhesion is desired a layer of biopolymer matrix.

One embodiment of the present invention provides a biomedical device coated with the biopolymer matrix to improve the biocompatibility thereof.

One embodiment of the present invention provides a process of preparing the biopolymer matrix disclosed in the present invention, wherein the process comprising cross linking chitosan hydrochloride and DDA in the presence of phosphate buffered saline.

One embodiment of the present invention provides a kit for a surgical and/or therapeutic use comprising the biopolymer matrix disclosed in the present invention.

The lack of water-solubility of commercial chitosan at neutral pH values complicates its use in pharmaceutical applications. The biological effects of chitosan are only effective at physiological pH value. In the present invention, chitosan hydrochloride in aqueous medium (pH 4-5) was employed to obtain an irreversible gel with dextran dialdehyde (DDA).

Dextran has well defined and repetitive chemical structure, good water solubility, low pharmacological activity and toxicity, presence of numerous reactive hydroxyl groups that allow derivatization. Thus, dextran finds wide application in biomedical field as drug delivery vehicle, wound dressings etc. In the present invention, dextran-dialdehyde (DDA) was used to prepare surgical bioadhesive gel.

The present invention aims at the development of a rapidly gelling polymeric system based on at least two natural polysaccharides, namely, chitosan and dextran which would find a number of biomedical uses such as tissue adhesive, wound dressings, as aneurysm filler, as an embolic agent, as a hemostatic agent, as a matrix for the controlled and prolonged delivery of drugs, growth factors, therapeutic proteins and peptides. The invention embodies the formation of a crosslinked three dimensional matrix by Schiff's reaction between oxidized dextran and chitosan hydrochloride at physiological pH conditions, and avoids the use of toxic crosslinking agents such as carbodiimide, glutaraldehyde, formaldehyde etc.

It was observed that chitosan (2% solution in 5% acetic acid, pH=3.1) on treatment with periodate-oxidized dextran (hereinafter termed as dextran dialdehyde, abbreviated as, DDA) forms gel which on standing goes back to solution. But on subsequent treatment with sodium borohydride, the gel becomes stable. It was also observed that when chitosan hydrochloride in aqueous medium (pH 4-5) with DDA was employed, an irreversible gel was obtained.

The present invention discloses the formation of a cross linked three dimensional matrix by Schiff's reaction between oxidized dextran and chitosan hydrochloride at physiological pH conditions, and avoids the use of toxic crosslinking agents such as carbodiimide, glutaraldehyde, formaldehyde etc.

The invention provides an adhesive composition for bonding biological tissues, including living tissues, to one another or for wound care management. This does not exhibit any toxicity risks, in particular due to diffusion of any crosslinking agent. The gel formation can be modified to take place rapidly (within a few seconds) which would facilitate its use as an injectable bioadhesive, aneurysm filler, embolic agent for blocking a blood vessel, in situ forming wound dressing etc.

The gel forming composition of the present invention can also be used to fabricate foams which absorb large amount of the wound exudates due to their macroporous nature. The fabrication of such foams can be done by passing air, nitrogen or an inert gas such as helium through the chitosan hydrochloride solution under agitation and then adding the DDA to crosslink the same. The foams thus produced can be shaped as sheets, rods, plugs, pads, etc., and can be used in the hydrated, semi-hydrated or dry form suitable for application in the wound site.

Periodate oxidation was used to introduce aldehyde group to the polysaccharide. Each ∝-glycol group consumes one molecular proportion of periodate, and, under given conditions, the rate of the reaction is dependent principally on the stereochemistry of the ∝-glycol group. The reaction produces dialdehyde residues in the polysaccharide. The extent of oxidation depends on the concentration of the reagents, substrate, time and temperature of the reaction and the molecular weight of the substrate. The oxidizing agent utilized for oxidation as aforesaid is periodic acid, more preferably, sodium or potassium periodate. Other reagents for introducing aldehyde functions to the polysaccharides include lead tetra acetate in an organic solvent such as dimethyl sulfoxide. After oxidation, purification and separation of the dialdehyde derivative of dextran from low molecular weight reaction components can be done by using dialysis membranes, precipitation, ultrafiltration or gel permeation chromatography, followed by lyophilization.

Chitosan salts can be obtained by the direct action of acids on chitosan dispersed in an organic medium. These chitosan salts are then used for crosslinking with any dialdehyde derivatives of polysaccharides like dextran, alginic acid etc. These solid chitosan salts or complexes, soluble in water, offer advantages of convenience, ease of control and simplicity in handling.

While various embodiments and/or individual features of the present invention have been illustrated and described, it would be obvious to those skilled in the art that various other changes and modifications can be made without departing from the spirit and scope of the invention. As will be also be apparent to the skilled practitioner, all combinations of the embodiments and features taught in the foregoing disclosure are possible and can result in preferred executions of the present disclosure.

### EXAMPLES

The following examples are put forth so as to provide those of ordinary skill in the art with a complete disclosure and the description of how to make and use the present invention, and are not intended to limit the scope of what the inventors regard as their invention nor are they intended to represent that the experiments below are all and only experiments performed.

### Example 1

### Preparation of Chitosan Hydrochloride

The chitosan hydrochloride was prepared by the method of Austin and Sennett, 1986. Chitosan (Viscosity average molecular weight 311 kDa, degree of deacetylation 74%) 10 g was dispersed in 100 mL of 60% ethanolic HCl. It was then kept stirring magnetically for 3 h at 20° C. The Chitosan hydrochloride formed was then filtered off and washed extensively with acetone-water mixture (6:2) until the filtrate was free from chloride ions as evidenced by lack of any precipitate with silver nitrate solution. The product was then dried at room temperature. The approximate yield of chitosan hydrochloride was 14 g (0.88 mole acid per mole Chitosan). The pH of a 10% solution of this modified chitosan in water was found to be between 4.5 and 5.

### Example 2

### Preparation of DDA

Dextran (5 g, M.W 500 kDa) was dissolved in 100 mL of distilled water. Calculated amount of sodium periodate was added to this solution according to the percentage of oxidation required. The solution was allowed to stir magnetically at 25° C. in dark for 6 h. The degree of oxidation was determined by iodometry. The solution was then dialyzed against distilled water until it was free from periodate. Complete removal of periodate was ensured by testing the dialyzate for the absence of turbidity or precipitate with an aqueous solution of silver nitrate. The solution was then frozen −78° C., lyophilized and stored in a desiccator in the refrigerator at 4° C. Representative data are given in Table 1. Yields ranged from 80 to 90%.

TABLE 1
Oxidation of dextran (MW 500,000 Daltons) with sodium metaperiodate

| Dextran | | |
|---|---|---|
| (gm) | Sodium m-periodate (gm) | Degree of oxidation (%) |
| 5 | 1.33 | 5.16 ± 0.2 |
| 5 | 3.35 | 50.14 ± 0.5 |
| 5 | 6 | 90.4 ± 0.43 |

### Example 3

### Preparation of Biopolymer Matrix (Gel) Comprising Chitosan Hydrochloride and DDA

DDA of different percent oxidations was made to react with chitosan hydrochloride to form the crosslinked gel. Gelation reaction was carried out in the presence of phosphate buffered saline (pH 7.4, 0.1 M). One mL of DDA (10% solution in phosphate buffered saline) was taken, to which 1 mL of chitosan hydrochloride (10% solution in water) added in a 15 ml vial (diameter 26 mm) and stirred using a Teflon magnetic stir bar (diameter 5 mm, length 10 mm at 50 rev/min). Gelling time was noted as the time required for the stir bar to stop using a stop watch according to Mo et al (X Mo, H Iwata, S Matsuda, Y Ikada, Soft tissue adhesive composed of modified gelatin and polysaccharides, J. Biomater. Sci. Polym. Ed, 2000, 11, 341-351). All the gelling experiments were carried out at 37° C. The gelling time obtained for all gels were within 3-6 seconds (see Table 2). There was no variation in gelling time irrespective of the extent of oxidation or the concentration of DDA employed.

TABLE 2
Gelling time of chitosan hydrochloride with DDA of different degree of
oxidation

| MW of | | DDA | | |
|---|---|---|---|---|
| Dextran | Oxidation | Conc in | Chitosan HCl | Gelling Time* |
| (KDa) | (%) | PBS (%) | Conc in Water (%) | at 37° C. (sec) |
| 500 | 5 | 5 | 10 | 3-4 |
| 500 | 50 | 10 | 10 | 3-4 |
| 500 | 90 | 15 | 10 | 3-4 |
| 500 | 5 | 5 | 5 | 3-6 |
| 500 | 50 | 10 | 5 | 3-4 |
| 500 | 90 | 15 | 5 | 3-6 |

### Example 4

### Analysis of Properties of the Biopolymer Matrix

### Viscosity Measurements

Viscosities of chitosan and DDA solutions were measured using a Viscometer in Small Sample Adaptor at 37° C. The viscosities of the chitosan solutions measured are shown in Table 3. The viscosities of solutions up to 15% are believed to be suitable for application using a syringe needle of 20-22 gauge.

TABLE 3
Viscosities of chitosan hydrochloride and DDA solutions

| | | Revolutions per | |
|---|---|---|---|
| Sample | Conc (%) | minute (RPM) | Viscosity (cP) |
| Chitosan HCl | 5 (in water) | 50 | <1 |
| Chitosan HCl | 5 (in water) | 100 | 6.2 |
| Chitosan HCl | 10 (in water) | 50 | 49.2 |
| Chitosan HCl | 10 (in water) | 100 | 59.4 |
| Chitosan HCl | 15 (in water) | 50 | 88.8 |
| Chitosan HCl | 15 (in water) | 100 | 92.7 |
| DDA (50% oxidized) | 10 (PBS) | 50 | 3.6-4.2 |

### Rate of Swelling of the Gels in Phosphate Buffered Saline (PBS)

One half mL 10% DDA (5. 50, 90% oxidized) and one half mL chitosan hydrochloride solution (10% solution in water) were mixed using a vortex mixer in a glass vial of 15 mL capacity and allowed to form gel of approximately 26 mm diameter and 20 mm thickness. It was then kept for 10 min at 37° C. and 5 mL of phosphate buffered saline (0.1 M, pH 7.4) was added to the gel and incubated the same at 37° C. At regular intervals of time, the weight of the gel was noted after removing PBS using Pasteur pipette. The percentage swelling was calculated based on the initial weight of the gel and its swollen weight as:

$$Swelling ( \% ) = \frac{Weight of swollen gel - Dry weight of gel \times 100}{Dry weight of gel}$$

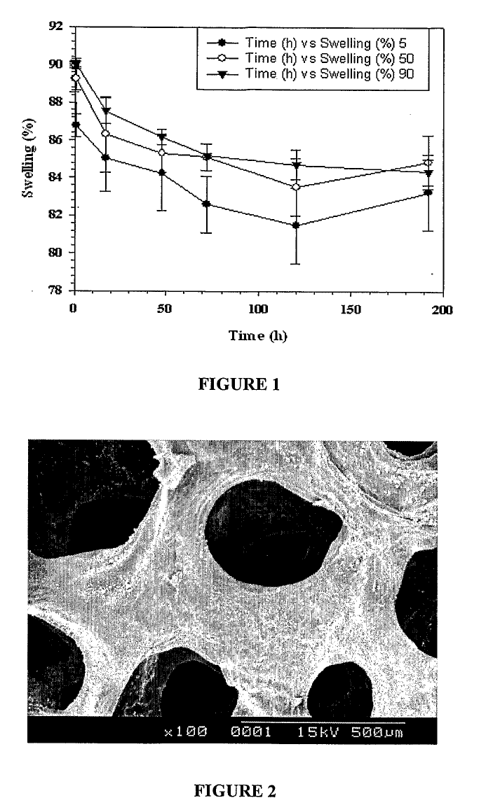

The rate of swelling of the gels prepared from a 10% solution of chitosan hydrochloride in water and 10% solution of DDA of different degrees of oxidation (5, 50 and 90%) in PBS is given in FIG. 1. Initially the gels have about 90% water; continued equilibration in PBS decreases the percentage of swelling slightly. The swelling is slightly decreased with respect to time since the medium is slightly alkaline and some amino groups of chitosan will be in the un-protonated form leading to reduced swelling. This is interesting from the point of view of the intended application as continued swelling in the presence of body fluids such as blood is not desirable for the application of the material as a surgical adhesive.

The cross linking density and molecular weight between crosslinks determined by swelling studies are shown in Table 4. As can be seen, when the concentration of chitosan is increased, it results in increased crosslinking density and lower molecular weight between crosslinks since the number of amino functions that can enter into Schiff's reaction with the aldehyde functions increases with increase in concentration of chitosan as expected.

TABLE 4
Crosslinking parameters of the hydrogel from chitosan-HCl and DDA

| | Crosslinking | |
|---|---|---|
| | density (υe × | Molecular weight between |
| Sample | 105) mol/cm3 | crosslinks, Mc (g/mol) |
| 5% Chitosan HCl + 10% | 6.80 ± 1.23 | 9159.37 ± 61.5 |
| DDA (MW 500 kD) | | |
| 10% Chitosan HCl + 10% | 21.81 ± 0.93 | 3231.09 ± 20.64 |
| DDA (MW 500 kD) | | |

### Surface and Internal Morphology

Surface and internal morphology of DDA/chitosan gels were examined by scanning electron microscopy (SEM). Lyophilized gels were cut using a razor blade to expose the inner region, placed on double-sided tape, sputter coated with gold and examined in the microscope for internal structure. The internal structure of the gel is shown in FIG. 2, exhibiting highly porous structure. These are lyophilized gels and not hydrated gels and the hydrated gels will have a different structure. However, these results are a pointer to the porous nature of these hydrogels.

### Cytotoxicity Evaluation

In vitro cytotoxicity testing was done using the direct contact method with the test sample based on ISO 10993-5 standards. Chitosan-HCl was crosslinked with DDA in PBS. Gels were lyophilized and subjected to cytotoxicity tests. Gels were also washed with water, lyophilized and then subjected to tests. The cytotoxicity tests using cell culture overlay using L929 mouse fibroblasts cells showed that the gels were non toxic in the unwashed and washed forms.

### Bonding Strength of Gels to Rat Skin

Rat skin was used to measure the bonding strength of the gel. The fatty portion of rat skin was removed using a scalpel and was cut into two pieces of 1×3 cm². One drop of chitosan hydrochloride solution (5% solution of chitosan hydrochloride in water was employed) was spread over the dermal side of one of the skin slices and one drop of DDA solution (10% in PBS) on the other slice. The two skin slices were then overlapped with a bonding area of 1×1 cm². After loading a weight of 50 g on the slices for regular intervals of time, tension required to peel off the skin patches was measured by connecting one skin slice to a pulley and other to a basket to which weights can be added, through a non-absorbable surgical suture; B Casali, 1992 (B Casali, F. Rodeghiera, A. Tosetto, B. Palmieri, R. Immovilli, C. Ghedini and P. Rivas.

Fibrin glue from single-donation autologous plasmapheresis. Transfusion. 1992; 32: 641-643). Bonding strength was measured as the load required for the glued skin to peel off by adding standard weights. Bonding strength was studied by varying the setting time and employing DDAs with different percentage oxidation. Each experiment was performed at least six times. Representative data are illustrated in the example shown in Table 5.

TABLE 5
Bonding strength of chitosan HCl-DDA glue to rat skin in vitro

| Oxidation of DDA (%) | Setting time (min)* | Bonding Strength (gf/cm2)† |
|---|---|---|
| 90 | 5 | 361 ± 44 |
| 50 | 5 | 250 ± 63 |
| 5 | 5 | 229 ± 70 |
| 90 | 10 | 363 ± 71 |
| 50 | 10 | 268 ± 80 |
| 5 | 10 | 247 ± 90 |

5% solution of chitosan HCl in water and 10% solution of DDA in PBS
*Bonding strength of Bioheal ® Fibrin Glue on Mouse skin: 45 and 50 gf/cm2 after setting time of 5 & 10 min. (Otani et al J. Biomed. Mater. Res., 31: 157, 1996).
†Average of 5 to 6 measurements

### Example 5

### Burst Test Experiments Using Rat Skin

A burst test was performed to examine the efficacy of the system as a tissue sealant, using a custom designed apparatus similar to the one reported by Prior et al with slight modifications (JJ Prior, Wallace D G, A Harner, N Powers; A hemostatic sealant formulation containing fibrillar collagen, bovine thrombin and autologous blood plasma. Ann Thorac Surg; 1999; 68: 479-485). To establish a uniform surface for testing the strength of the gel and its adhesion to a test substrate, a syringe pump (Master Flex) was utilized consisting of pressure gauge connected by fluid filled tubing to a circular sample plate with a central orifice of 2 mm in diameter. To simulate a tissue surface, the sample plate was covered with rat skin, fastened to the plate by a gasket seal. The skin also had 2 mm hole pierced in it, but offset from the hole in the sample plate. The sheet was moistened with 0.9% aqueous NaCl and placed on the plate. The test formulation was sprayed (5% chitosan hydrochloride in water and 10% DDA in PBS, 40 μL each) on to the tissue surface and allowed to gel for about 5 minutes (see Table 6) and then the tubing containing phosphate buffered saline (PBS pH 7.4) was pressurized by use of a syringe pump. Pressure in the line was measured on the pressure gauge and the pressure at which water burst through the gel was recorded. The experiments were done in triplicate.

TABLE 6
Burst strength of DDA-chitosan adhesive

| Oxidation of DDAMW 500 kDa | | Burst Strength¶ |
|---|---|---|
| (%) | Setting Time (min) | (mm Hg)† |
| 90 | 5 | 361 ± 44 |
| 50 | 5 | 250 ± 63 |
| 5 | 5 | 229 ± 70 |

¶Prior et al Ann Throac Surg 68: 479, 1999.
5% solution of chitosan HCl and 10% solution of DDA in PBS
†Average of 5 to 6 measurements

### Example 6

### In Vivo Evaluation of the Adhesive in Rabbit Liver Parenchymal Injury Model

The DDA-chitosan hydrochloride adhesive system was evaluated for its performance by examining its haemostatic effect on liver injury and safety by studying tissue response at 14 days. The experimental design consisted of a rabbit liver injury model under normal physiological conditions. Clinically proven biosynthetic, albumin glutaraldehyde based glue (Bioglue™, CryoLife Inc., USA) was used as control. A total of 6 animals were used for the study with 3 injuries in one animal. DDA (50% oxidized) and chitosan hydrochloride in the solid form were ETO (Ethylene oxide) sterilized using standard protocols and solutions of appropriate concentrations were prepared in sterile media (PBS or water).

New Zealand white rabbits weighing 3-4 kg were employed in the study. A total of 6 animals were used for the study. Animals were fed with standard rabbit pellets and water ad libitum. Test glue was applied in 3 animals and control glue was applied in the remaining three. Under general anaesthesia (Ketamine and thiopentone sodium, controlled on dorsal recumbence), the ventral abdomen of the animals was draped for aseptic surgery. Liver was assessed by a right paracostal incision of nearly 4 to 5 cm length. The following types of injuries were made on the liver.
Liver lobe: Liver lobe edge resection of approximately 1.5 cm length at two sites.
Liver lobe circular excision of approximately 1 cm diameter at one site.

The site was cleaned free of blood. Test/control glue was applied and examined for haemostasis. The two component test glue was applied serially. First, 0.5 mL of DDA was applied followed by 0.5 mL of chitosan-HCl using two separate syringes. Control glue was applied using the applicator provided by the manufacturer. The abdomen was closed as routine. Analgesics (Tidigesic) and antibiotic (Tetracycline) coverage was given. The animals were returned to their individual cages and observed daily for any adverse clinical symptoms. The animals were sacrificed at the end of 14 days and tissue response was studied on paraffin sections. As the components of the test glue were applied one by one in separate syringes there was no possibility of clogging as the components only mix at the site of incision. First application of DDA seemed to minimise the bleeding. After the application of chitosan-HCl, total stoppage of bleeding was noticed in both edge resection and circular excision sites. In the case of control glue, the glue has to be applied within 30 to 40 seconds, before the delivery tip is clogged. Assembling the delivery gun following autoclave sterilization was found to be extremely difficult. The piston was stuck and on application force, the piston tip broke. Also, the control glue was less transparent and less tissue compliant compared to the test glue.

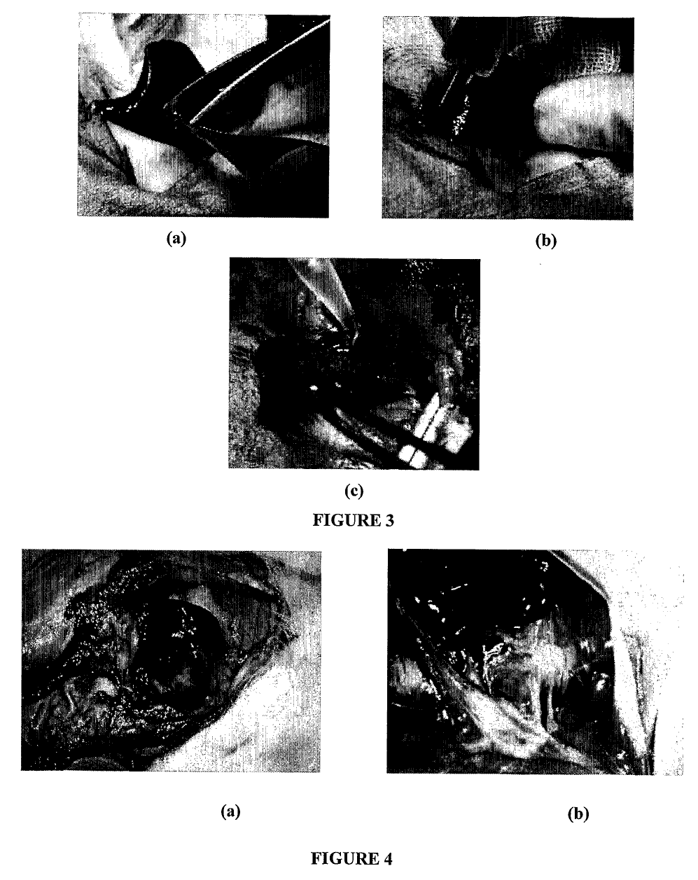

FIGS. 3 (a) and (b) show the creation of rabbit liver injury and application of the test glue respectively and FIG. 3 (c) shows the application of control glue.

FIG. 4 (a) shows the test glue at 14 days and FIG. 4 (b) shows the control glue at 14 days. The adhesion of liver into abdominal wall in both control and test glue was seen in all animals.

Histologically, at the end of two weeks, in the case of control glue, a thick layer of glue was seen as pink homogenous material. Necrosis was noticed directly beneath the glue surrounded by inflammation (pentacle) consisting of macrophages, lymphocytes and eosinophils. Giant cells were noticed near the glue. Subcapsular inflammation and fibrosis was also noticed.

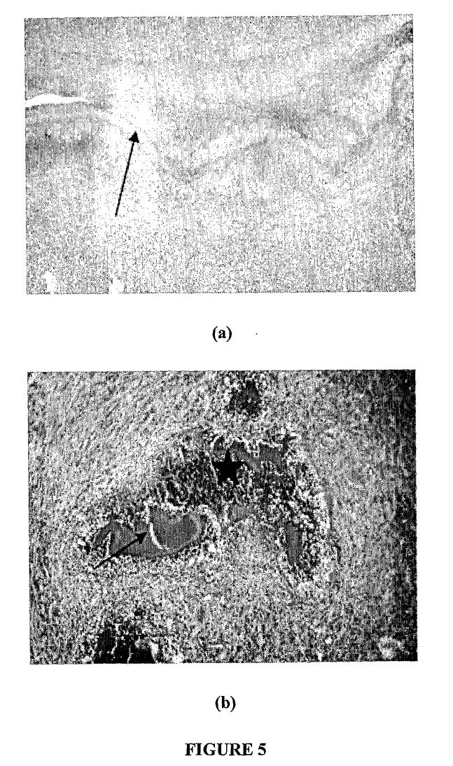

In the case of test glue, a thin layer of glue was seen as pink homogenous material (FIG. 5a, arrow). Adjacent to glue the area of necrosis (star), inflammation and fibrosis noticed was less compared to control. Some giant cells and macrophages were also noticed (FIG. 5b). The capsular thickening and fibrosis seen beneath the capsule affecting superficial parenchyma was observed. Localized suppurative necrosis near glue was also noticed.

In summary, the test and control glue could effectively control the haemostasis of liver injury. But, the tissue response such as necrosis, inflammation and fibrosis was comparably less in case of test glue in comparison to the control glue.

### Example 7

### In Vivo Evaluation of the DDA-chitosan Hydrochloride Adhesive in Sheep Lung Parenchymal Injury Model

The DDA-chitosan hydrochloride adhesive was also evaluated for its performance and safety in sheep lung injury model. In the experimental design, the glue was tested in sheep lung injury model under normal physiological as well as under coagulopathic conditions (animal heparinized with activated clotting time more than twice the physiological value). Bioglue™ was used as control glue. A total of 8 animals were used in the study, 4 animals for 14 days and 4 animals for 3 months duration. Each animal had four sites of injury on the right lung. Test and control glue were applied on 2 animals for each duration with each animal giving 4 sites of application. As before, appropriate concentrations of ETO-sterile DDA and chitosan HCl were prepared in sterile media (PBS or water).

Under general anaesthesia, the right lateral thorax of the animal was draped for aseptic surgery. Thoracotomy was done through 5th intercostal space. Animal was heparinized. The following sites on the diaphragmatic and middle lobe of lung were identified and incisional injuries of approximately 5 mm depth and 4 cm length were made using scalpel on the following sites. Diaphragmatic lobe: Superior site; Diaphragmatic lobe: Inferior site; Middle lobe: Superior site and Middle lobe: Inferior site. Incisions were made on the identified lung sites under inflation with ambu bag (peak air way at 20 mm of Hg). Leaking of air and blood from the site were confirmed. Ventilation was stopped and the site was cleaned free of blood and test/control glue was applied on all the four sites. The control glue was applied as per manufacturer's instruction using the gun provided. The test glue containing, 1 mL of DDA followed by 1 mL of chitosan-HCl using two separate syringes was applied and subsequently mixed on the site of injury. Ventilation was resumed immediately. The incision site was observed for air and blood leak.

The chest was closed as routine. Heparin was reversed using protamine sulphate. Chest tube had to be maintained more than 45 min. Blood collection in the chest drain was noted. Chest tube was removed when blood draining was nearly nil. The wound was dressed and the animal was extubated. Animals were returned to their individual pens and fed with standard sheep feed and water ad libitum and they were daily observed for any adverse clinical symptoms. Analgesics and antibiotic coverage were given as usual.

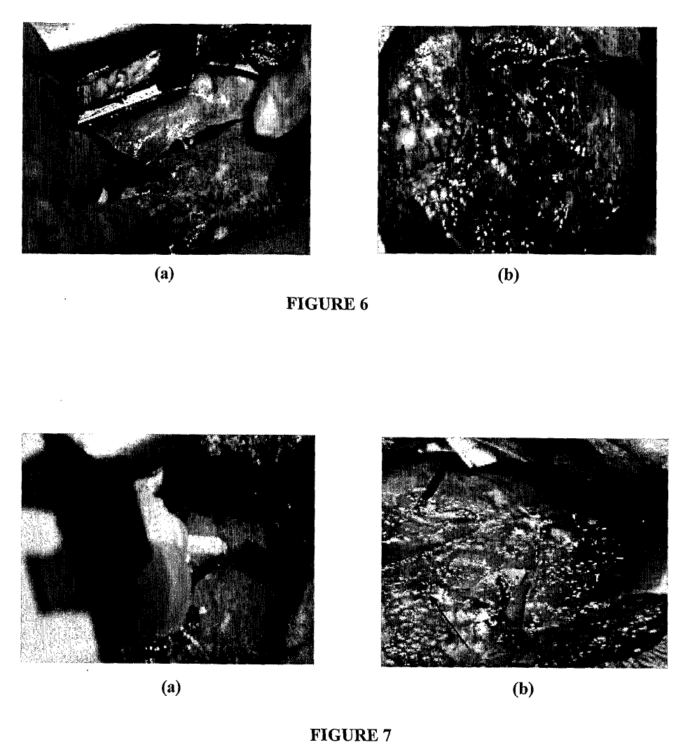

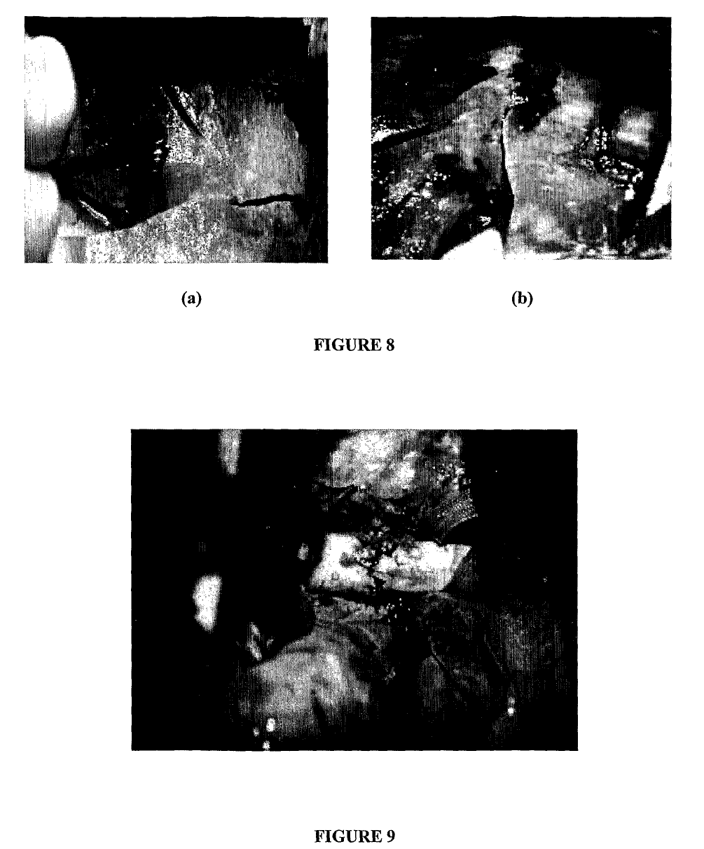

FIG. 6 shows the creation of lung injury. Air and blood leak from incision site were noted. After application of the control glue, persistent air leak was noticed from the incision site (FIG. 7). Application of the test glue (DDA-chitosan HCl) resulted in complete sealing of the site and there was no air leak observed in any of the animals (FIG. 8).

Histopathologically, at the end of 14 days, the test glue can be identified as pink homogenous material. A zone of inflammation and fibrosis noticed around the glue. Macrophages, lymphocytes, a few polymorphonuclear cells and giant cells were noticed in the inflammatory zone. Mild thickening of pleura, sub-pleural fibrosis and thickening were noticed.

In the case of control, the glue is identified as pink homogenous material but fibrosis and inflammation were seen encircling the glue. Moderate inflammation consisting of macrophages, lymphocytes, giant cells and polymorphonuclear cells were noticed. Thickening of pleura and sub-pleural thickening were also seen. Areas of suppurative inflammation and necrosis in contact with the glue are also seen.

At the end of three months, in the case of test glue, remnants of glue were still noticed. Fibrosis and mild inflammation with infiltration of macrophages were also noticed adjacent to glue. Resorption of test material by giant cells is seen. Lung parenchyma is seen unaffected by the glue.

In summary, the test glue was more successful in sealing the lung injury compared to the control glue.

### Example 8

### In Vivo Evaluation of the Adhesive in a Pig Arterial Injury Model

The objective of the study was to evaluate the sealing ability of the test glue as an adjunct to sutures in standard aortic incisions and to examine the tissue response elicited by the glue at 2 weeks period. The evolution of aortic incision healing was studied microscopically. The sealing ability of the test glue was assessed by observing for presence of blood leak from the site of apposition-sutured aortic incision following glue application. Confirmation of blood leak from the apposition-sutured aortic incision in the same animal before glue application acted as control. The safety of the glue was studied by observing the tissue response which consists of evaluation of degenerative, necrotic, inflammatory and proliferative response of the vascular tissue at 2 weeks. Examined during the course of this investigation were the complete sealing of aortic incisions, any other complications at recovery, observation of animal for any adverse clinical outcome during the post surgical period and observation of aortic aneurysms at termination and histopathological evaluation at 2 weeks. As before, appropriate concentrations of ETO (Ethylene Oxide) sterile DDA and chitosan HCl were prepared in sterile media (PBS or water).

Eight miniature adult swine weighing 40-70 kg of either sex were employed in the study. Animals were housed in individual cages at ambient temperature under natural lighting. Standard pig feed, with green and clean tap water were given ad libitum.

Under general anesthesia, on right lateral recumbence and total asepsis, left lateral thoracotomy was made at 5th intercostal space. The azygous vein was ligated and excised off exposing the thoracic aorta. Under heparinization, the aorta was clamped and a transverse aortic incision of 10 to 15 mm was made. This incision was apposed with 2 to 4, 4/0 prolene sutures. The clamp was removed briefly to ascertain bleeding from the sutured aortic incision. Clamps were then re-applied. The site was dried using surgical gauze and glue was applied. 1 mL of test glue was applied first followed by two applications at 30 seconds interval. Thus, the test glue was applied using the provided dispenser (a double syringe fibrin glue applicator) in three layers. There was one thoracic aortic incision in each animal.

After a period of 2 min, the clamps were removed and observed for bleeding from the applied site. On complete sealing of the incision site, the chest was closed as routine. Animals were given antibiotic and analgesics for first five post operative days. They were observed daily for any clinical abnormality up to one week postoperatively and then periodically.

At the end of the study period which was 2 weeks, animals were euthanized by an excess dose of intravenous thiopentone sodium. The thorax was examined for tissue adhesions, aneurysms of aorta over the incision site or for any other significant gross observations. The thoracic aorta containing the glue applied site (as identified by the presence of sutures) was excised and part of it was cryo-preserved and rest was immersion fixed in 10% buffered formalin. The mediastinal lymph node was collected and cryo-preserved for immuno-histochemistry observations.

The fibrin glue applicator was used in the study. A 5% solution of chitosan hydrochloride in water and a 10% solution of DDA in 0.1 M PBS were prepared and kept at 37° C. in water bath in sterile polypropylene centrifuge tubes. Solutions were aspirated into the syringes before application. The gelation time of the two-component glue was tested in an Actalyke ACT tester in G-ACT tubes in every time before the solution was applied in order to assess the gelling time.

The gelation observed by in vitro using magnetic stirrer, the rotation of the tube in the mixer took a slightly longer time for (20-25 sec) for complete gelation. Even solutions stored over two or three days were found to show similar gelation times and therefore fresh solutions were not prepared before each and every experiment. The glue could seal effectively the standard aortic incision in all the eight cases (FIG. 9). The glue could effectively seal the aortic incision in first five cases with single round of application. However, in last three cases up to three rounds of glue application were required for complete sealing.

All the animals survived the surgery and completed the duration of study uneventfully. No adverse effects of clinical significance were reported during the period of the study. On autopsy at the end of two weeks, the aortic sealings were intact in all the eight cases. There were tissue adhesions over the glue applied area. There were no incidences of aneurisms in any of the cases.

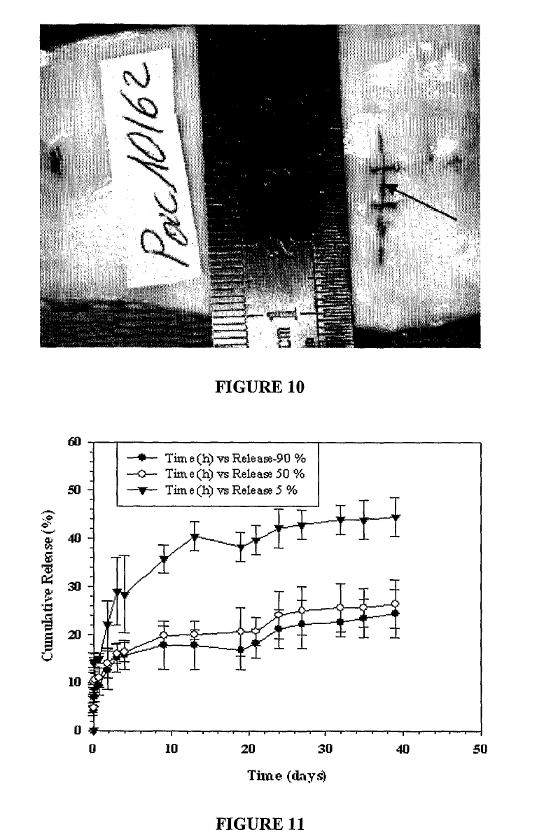

The endoluminal surface of the sealed incision in all the cases showed intact apposition with adequate healing across the incision. No tissue necrosis or inflammation of endothelial surface could be seen grossly (FIG. 10).

The test glue could be used in high pressure areas like thoracic aorta. The glue could seal the incisions effectively and the sealing was retained even at the end of 14 days by which time natural healing process will strengthen the incision site. Thus, it is concluded that the test glue as compared to the control glue is highly effective adhesive biomaterial for sealing incisions.

### Example 9

### Controlled Release of FITC-Albumin from the Gel

To study the release profile of high molecular weight proteins, different types of gels were loaded with FITC-labeled bovine serum albumin (2.5% loading) and cumulative release was followed. Briefly, 0.15 mL of DDA (10% solution in PBS, pH 7.4, 0.1M) was taken in a screw-capped test tube, to which was added 0.15 mL of chitosan hydrochloride (10% solution in water) containing FITC-albumin (2.5% loading) and allowed to form gel. It was then kept at 37° C. for 10 min and 10 mL of PBS was then introduced and incubated at 37° C. At regular intervals, 1 mL aliquots were withdrawn and absorbance of released FITC-albumin was read at 496 nm in a UV-Visible spectrophotometer. Cumulative release was then calculated. All the experiments were done in triplicate. Representative data are illustrated in the example shown in FIG. 11.

FITC-albumin release from all gels was slow and lasted over many days (FIG. 11). At the end of 39 days, only about 25% was found to be released from gels prepared from 50 and 90% oxidized DDA, while about 40% was released from gels cross linked with 5% DDA. The release from gels having degree of oxidation 50% and 90% were slowed down due to the highly cross linked nature of matrix as well as better protein conjugation due to the availability of more aldehyde functions. The release reached almost an asymptotic phase at the end of 40 days and possibly more will released when the material undergoes further biodegradation. This investigation shows that the system will be suited for controlled release of therapeutic peptides and proteins.

### Controlled Release of 5-Fluorouracil

Gels prepared using DDA of different degree of oxidation were loaded with an anticancer drug, 5-fluorouracil at different loadings (2.5%, 5% and 10%) and cumulative release was followed. Briefly, 0.15 mL of DDA (10% solution in PBS) was taken in a screw-capped test tube, to which was added 0.15 mL of chitosan hydrochloride (10% solution in water) containing different amounts of 5-fluorouracil and allowed to form gel. It was then kept at 37° C. for 10 min, 10 mL of PBS was then introduced and incubated at 37° C. At regular intervals, 1 mL aliquots were withdrawn and absorbance of released 5-fluorouracil was read at 265 nm in a UV-Visible spectrophotometer. Cumulative release was then calculated. All the experiments were done in triplicate. Representative data are illustrated in the example shown in Table 7.

TABLE 7
Cumulative release of 5-Flurouracil

| | Cumulative Release (%) | | |
|---|---|---|---|
| | DDA (90% | DDA (50% | DDA (5% |
| Time (h) | oxidized) | oxidized) | oxidized) |
| 0.5 | 20.9 ± 6 | 47.0 ± 3 | 33.3 ± 2 |
| 1 | 24.3 ± 6 | 47.7 ± 6 | 40.7 ± 3 |
| 2 | 37.4 ± 5 | 71.2 ± 6 | 72.4 ± 1 |
| 3 | 55.9 ± 3 | 87.1 ± 3 | 81.7 ± 2 |
| 4 | 70.8 ± 6 | 89.3 ± 1 | 86.9 ± 0.4 |
| 5 | 82.1 ± 4 | 91.8 ± 3 | 86.6 ± 0.8 |
| 6 | 85.8 ± 5 | 91.8 ± 2 | 91.8 ± 5 |
| 8 | 91.3 ± 5 | 92.1 ± 2 | 92.1 ± 2 |
| 10 | 91.3 ± 5 | 92.1 ± 2 | 92.1 ± 2 |

### Example 10

### Preparation of Biopolymer Matrix (Gel) Comprising Chitosan Acetate and DDA

DDA of different percent oxidations was made to react with chitosan acetate to form the crosslinked gel. Gelation reaction was carried out in the presence of phosphate buffered saline (pH 7.4, 0.1 M). One mL of DDA (10% solution in phosphate buffered saline) was taken, to which 1 mL of chitosan acetate (10% solution in water) added in a 15 ml vial (diameter 26 mm) and stirred using a Teflon magnetic stir bar (diameter 5 mm, length 10 mm at 50 rev/min). Gelling time was noted as the time required for the stir bar to stop using a stop watch. All the gelling experiments were carried out at 37° C. The gelling time obtained for all gels were within 3-6 seconds. There was no variation in gelling time irrespective of the extent of oxidation or the concentration of DDA employed.

### Example 11

### Preparation of Biopolymer Matrix (Gel) Comprising Chitosan lactate and DDA

DDA of different percent oxidations was made to react with chitosan lactate to form the crosslinked gel. Gelation reaction was carried out in the presence of phosphate buffered saline (pH 7.4, 0.1 M). One mL of DDA (10% solution in phosphate buffered saline) was taken, to which 1 mL of chitosan lactate (10% solution in water) added in a 15 ml vial (diameter 26 mm) and stirred using a Teflon magnetic stir bar (diameter 5 mm, length 10 mm at 50 rev/min). Gelling time was noted as the time required for the stir bar to stop using a stop watch. All the gelling experiments were carried out at 37° C. The gelling time obtained for all gels were within 3-6 seconds. There was no variation in gelling time irrespective of the extent of oxidation or the concentration of DDA employed.

### Example 12

### Preparation of Biopolymer Matrix (Gel) Comprising Chitosan sulphate and DDA

DDA of different percent oxidations was made to react with chitosan sulphate to form the crosslinked gel. Gelation reaction was carried out in the presence of phosphate buffered saline (pH 7.4, 0.1 M). One mL of DDA (10% solution in phosphate buffered saline) was taken, to which 1 mL of chitosan sulphate (10% solution in water) added in a 15 ml vial (diameter 26 mm) and stirred using a Teflon magnetic stir bar (diameter 5 mm, length 10 mm at 50 rev/min). Gelling time was noted as the time required for the stir bar to stop using a stop watch. All the gelling experiments were carried out at 37° C. The gelling time obtained for all gels were within 3-6 seconds. There was no variation in gelling time irrespective of the extent of oxidation or the concentration of DDA employed.

1. A biodegradable biopolymer matrix for use as bioadhesive or tissue sealant comprising chitosan hydrochloride and a dialdehyde derivative of polysaccharide, wherein chitosan hydrochloride is in the range of about 1% to 20%, preferably 5% to 10%.

2-5. (canceled)

6. The matrix as claimed in claim 1, wherein said polysaccharide is dextran or alginic acid.

7. The matrix as claimed in claim 1, wherein said dialdehyde derivative of polysaccharide is dextran dialdehyde (DDA).

8. The matrix as claimed in claim 7, wherein concentration of said DDA is 1% to 10% preferably at a concentration of 10%.

9. (canceled)

10. The biodegradable biopolymer matrix as claimed in claim 1, wherein chitosan hydrochloride and dextran dialdehyde is in the ratio of 1:1 to 1:2.

11. The biodegradable biopolymer matrix as claimed in claim 1, wherein chitosan hydrochloride and dextran dialdehyde is in the ratio of 1:1.

12. (canceled)

13. A process of preparing the biopolymer matrix as claimed in claim 1, wherein said process comprising cross linking chitosan hydrochloride and DDA in the presence of phosphate buffered saline.

14. A method of sealing a tissue or wound in an animal comprising applying to said tissue or wound a matrix as a bioadhesive or a tissue sealant wherein said matrix comprises chitosan hydrochloride and a dialdehyde derivative of polysaccharide, wherein chitosan hydrochloride is in the range of about 1% to 20%, preferably 5% to 10%.

15. (canceled)

16. A drug delivery system comprising the biopolymer matrix as claimed in claim 1.

17. The method of claim 14, wherein said polysaccharide is dextran or alginic acid.

18. The method of claim 14, wherein said dialdehyde derivative of polysaccharide is dextran dialdehyde (DDA).

19. The method of claim 18, wherein concentration of said DDA is 1% to 10% preferably at a concentration of 10%.

20. The method of claim 14, wherein chitosan hydrochloride and dextran dialdehyde is in the ratio of 1:1 to 1:2.

21. The method of claim 14, wherein chitosan hydrochloride and dextran dialdehyde is in the ratio of 1:1.
